(19) United States
(12) Patent Application Publication
(10) Pub. No.: US 2025/0057576 A1
(43) Pub. Date: Feb. 20, 2025

(54) ADAPTERS FOR SURGICAL IMPACTING TOOLS

(71) Applicant: DePuy Synthes Products, Inc., Raynham, MA (US)

(72) Inventors: Nathanael Conner, Roanoke, IN (US); Cory Shulaw, Warsaw, IN (US)

(21) Appl. No.: 18/781,097

(22) Filed: Jul. 23, 2024

Related U.S. Application Data
(60) Provisional application No. 63/519,450, filed on Aug. 14, 2023.

(51) Int. Cl. A61B 17/92; A61B 17/00; A61F 2/46

(52) U.S. Cl. CPC A61B 17/92; A61F 2/4607; A61B 2017/00486; A61F 2002/4681

(57) ABSTRACT

Various exemplary adapters for surgical impacting tools and methods of using adapters for surgical impacting tools are provided. In general, an adapter can be configured to be releasably attached to a surgical implement configured to impact bone. The adapter can be fixedly, non-releasably attached to a surgical impacting tool handpiece or can be configured to be releasably attached to a surgical impacting tool handpiece. The surgical impacting tool handpiece, such as a handpiece of an orthopedic impactor, is configured to drive impacting of the surgical implement relative to bone. Each of a plurality of surgical implements configured to be attached to the adapter can be different from one another in one or more aspects.

### CROSS REFERENCE TO RELATED APPLICATIONS

The present application claims priority to U.S. Provisional Patent Application No. 63/519,450 entitled “Adapters For Surgical Impacting Tools” filed on Aug. 14, 2023, which is hereby incorporated by reference in its entirety.

### FIELD

The present disclosure relates generally to adapters for surgical impacting tools.

### BACKGROUND

In the field of orthopedics, prosthetic devices, such as artificial joints, are often implanted or seated in a patient's bone cavity. The cavity is typically formed during surgery before a prosthetic device is seated or implanted by, for example, a physician or other medical professional removing and/or compacting existing bone to form the cavity. The prosthetic device, which can also be referred to as a prosthesis, usually includes a stem or other protrusion that is inserted into the cavity.

To create the cavity, a physician or other medical professional may use a broach, chisel, or other surgical implement conforming to the shape of the stem of the prosthetic device. In general, the surgical implement is impelled into the implant area to form the cavity. One technique for impelling the surgical implement includes a physician or other medical professional manual hammering the surgical impacting tool to impel the surgical implement into the implant area. Another technique for creating the prosthetic cavity relies on computer-controlled robotic arms for creating the cavity instead of using manual power provided by a physician or other medical professional. Another technique for creating the prosthetic cavity is to drive the surgical implement pneumatically, e.g., by compressed air. Another technique for creating the prosthetic cavity relies on a linear compressor to compress air on a single stroke basis and then, after a sufficient pressure is created, to release the air through a valve and onto a striker to impel the surgical implement.

The broach, chisel, or other surgical implement can be removably coupled to the surgical impacting tool to, for example, allow for surgical implements of different sizes and/or shapes to be used with the surgical impacting tool in different surgical procedures to help accommodate a particular patient's needs, to allow for replacement of surgical implements that become worn, damaged, or otherwise undesirable for future use without having to replace a remainder of the surgical impacting tool, and/or to accommodate a surgeon's personal preference of surgical implements. However, various techniques for creating the prosthetic cavity that impel the surgical implement, such as the four techniques discussed above, can loosen the surgical implement's removable coupling to the surgical impacting tool due to the force required to impel the surgical implement. Such loosening may cause the surgical implement to unexpectedly become decoupled from the surgical impacting tool during a surgical procedure, may cause the surgical implement to shake or otherwise move in unintentional direction(s) and thus cause patient harm and/or adversely affect cavity formation, and/or may hinder cavity formation by not allowing the surgical implement to receive and be impelled at full intended force.

Accordingly, there remains a need for improved surgical impacting tools.

### SUMMARY

In general, adapters for surgical impacting tools and methods of using adapters for surgical impacting tools are provided.

In one aspect, a surgical device is provided that in one embodiment includes a first adapter and a second adapter. The first adapter is configured to extend distally from a surgical impacting tool handpiece configured to drive impacting of bone. The first adapter is configured to releasably attach to the surgical impacting tool handpiece, and the first adapter includes a first mating feature configured to releasably couple to a first mating element of a first surgical implement configured to impact bone. The second adapter is configured to extend distally from the surgical impacting tool handpiece. Only one of the first and second adapters is configured to extend distally from the surgical impacting tool handpiece at a time. The second adapter is configured to releasably attach to the surgical impacting tool handpiece, the second adapter includes a second mating feature configured to releasably couple to a second mating element of a second surgical implement configured to impact bone, and the second mating feature is different than the first mating feature.

The surgical system can vary in any number of ways. For example, the second mating feature can have a different geometry than the first mating feature.

For another example, the first mating feature can be configured to cooperate with the first mating element to prevent rotation of the first surgical implement relative to the first adapter with the first surgical implement releasably attached to the first adapter. In some embodiments, the first mating feature can include a distal-facing cavity having a cross pin extending across a diameter of the cavity. In some embodiments, the first mating feature can include a distally-extending protrusion configured to be seated in a proximal-facing cavity of the first surgical implement, the protrusion can have a cross-sectional shape that is one of D-shaped, ovular, Z-shaped, rectangular, star-shaped, hexagonal, or octagonal, and the proximal-facing cavity can have a corresponding cross-sectional shape. Further, the protrusion can extends from a face of the first adapter that is free of any cavities formed therein; and/or the second mating feature can be configured to cooperate with the second mating element to prevent rotation of the second surgical implement relative to the second adapter with the second surgical implement releasably attached to the second adapter, the second mating feature can include a second distally-extending protrusion configured to be seated in a proximal-facing cavity of the second surgical implement, and the second protrusion can have a cross-sectional shape that different than the cross-sectional shape of the protrusion of the first adapter. In some embodiments, the first mating feature can include a distal-facing cavity configured to receive therein a proximally-extending protrusion of the first surgical implement, the distal-facing cavity can have a cross-sectional shape that is one of D-shaped, ovular, Z-shaped, rectangular, star-shaped, hexagonal, or octagonal, and the protrusion can have a corresponding cross-sectional shape. Further, the distal-facing cavity can be formed in a face of the first adapter that is free of any protrusions extending distally therefrom; the second mating feature can be configured to cooperate with the second mating element to prevent rotation of the second surgical implement relative to the second adapter with the second surgical implement releasably attached to the second adapter, the second mating feature can include a second distal-facing cavity configured to receive therein a proximally-extending protrusion of the second surgical implement, and the second distal-facing cavity can have a cross-sectional shape that different than the cross-sectional shape of the distal-facing cavity of the first adapter; and/or the second mating feature can be configured to cooperate with the second mating element to prevent rotation of the second surgical implement relative to the second adapter with the second surgical implement releasably attached to the second adapter.

For another example, the first mating feature can be one of: a distal-facing cavity having a cross pin extending across a diameter of the cavity, a distally-extending protrusion configured to be seated in a proximal-facing cavity of the first surgical implement and having a cross-sectional shape that is one of D-shaped, ovular, Z-shaped, rectangular, star-shaped, hexagonal, or octagonal, and a distal-facing cavity configured to receive therein a proximally-extending protrusion of the first surgical implement and having a cross-sectional shape that is one of D-shaped, ovular, Z-shaped, rectangular, star-shaped, hexagonal, or octagonal.

For yet another example, the first adapter can also include a first pivotable arm configured to pivot into engagement with the first surgical implement releasably coupled to the first adapter, and the second adapter can also include a second pivotable arm configured to pivot into engagement with the second surgical implement releasably coupled to the second adapter. Further, the first pivotable arm can include one of (1) a textured surface configured to engage a surface of the first surgical implement to improve retention of the first adapter to the first surgical implement and (2) a magnet configured to magnetically attract to the first surgical implement to improve retention of the first adapter to the first surgical implement, and the second pivotable arm can include one of (1) a textured surface configured to engage a surface of the second surgical implement to improve retention of the second adapter to the second surgical implement and (2) a magnet configured to magnetically attract to the second surgical implement to improve retention of the second adapter to the second surgical implement.

For still another example, only one of the first and second adapters can include a pivotable arm configured to pivot into engagement with the surgical implement releasably coupled to the one of the first and second adapters, and the second adapter can also include a second pivotable arm configured to pivot into engagement with the second surgical implement releasably coupled to the second adapter. Further, the one of the first and second adapters can include one of (1) a textured surface configured to engage a surface of the surgical implement to improve retention of the one of the first and second adapters to the surgical implement and (2) a magnet configured to magnetically attract to the surgical implement to improve retention of the one of the first and second adapters to the surgical implement.

For another example, the first mating feature can be integrally formed with the first adapter, and the second mating feature can be either integrally formed with the second adapter or configured to releasably attach to the second adapter.

For still another example, the first mating feature can be configured to releasably attach to the first adapter, and the second mating feature can be either integrally formed with the second adapter or configured to releasably attach to the second adapter.

For another example, a proximal portion of the first adapter or a proximal portion of the second adapter can be configured to be received within the surgical impacting tool handpiece, the first mating feature can be on a distal-facing surface of the first adapter, and the second mating feature can be on a distal-facing surface of the second adapter.

For yet another example, the surgical system can include the surgical impacting tool handpiece. Further, the surgical system can include the first and second surgical implements; and/or the surgical impacting tool handpiece can include a distal-facing cavity configured to receive a first portion of the first adapter therein or a second portion of the second adapter therein, the first mating feature can be on a distal-facing surface of the first adapter, and the second mating feature can be on a distal-facing surface of the second adapter.

For still another example, the surgical system can include a third adapter configured to extend distally from the surgical impacting tool handpiece, only one of the first, second, and third adapters can be configured to extend distally from the surgical impacting tool handpiece at a time, the third adapter can be configured to releasably attach to the surgical impacting tool handpiece, the third adapter can include a third mating feature configured to releasably couple to a third mating element of a second surgical implement configured to impact bone, and the third mating feature can be different than the first mating feature and the second mating feature. Further, the surgical system can include the surgical impacting tool handpiece. Further, the surgical system can include the first, second, and third surgical implements.

In another embodiment, a surgical system includes a surgical impacting tool handpiece configured to drive impacting of bone, and an adapter configured to extend distally from the surgical impacting tool handpiece. The adapter includes a mating feature configured to releasably couple to a mating element of a surgical implement configured to impact bone. The mating feature is configured to cooperate with the mating element to prevent rotation of the surgical implement relative to the adapter with the surgical implement releasably attached to the adapter. The mating feature is one of: a distal-facing cavity having a cross pin extending across a diameter of the cavity, a distally-extending protrusion configured to be seated in a proximal-facing cavity of the surgical implement and having a cross-sectional shape that is one of D-shaped, ovular, Z-shaped, rectangular, star-shaped, hexagonal, or octagonal, and a distal-facing cavity configured to receive therein a proximally-extending protrusion of the surgical implement and having a cross-sectional shape that is one of D-shaped, ovular, Z-shaped, rectangular, star-shaped, hexagonal, or octagonal.

The system can have any number of variations. For example, the mating feature can be configured to cooperate with the mating element to prevent rotation of the surgical implement relative to the adapter with the surgical implement releasably attached to the adapter.

For another example, the mating feature can be integrally formed with the adapter.

For yet another example, the mating feature can be configured to releasably attach to the adapter.

For still another example, a proximal portion of the adapter can be configured to be received within the surgical impacting tool handpiece, and the mating feature can be on a distal-facing surface of the first adapter.

For another example, the surgical system can include the surgical implement.

For still another example, the adapter can be configured to releasably attach to the surgical impacting tool handpiece. Further, the surgical system can include at least one additional adapter, each of the at least one additional adapters can be configured to releasably attach to the surgical impacting tool handpiece such that only one of the adapter and the at least one additional adapter is configured to extend distally from the surgical impacting tool handpiece at a time, each of the at least one additional adapters can include a second mating feature configured to releasably couple to a second mating element of a second surgical implement configured to impact bone, and the second mating feature can be configured to cooperate with the second mating element to prevent rotation of the second surgical implement relative to the additional adapter with the second surgical implement releasably attached to the second adapter.

For another example, the adapter can be non-releasably attached to the surgical impacting tool handpiece.

In another embodiment, a surgical system includes a first adapter and a second adapter. The first adapter is configured to extend distally from a surgical impacting tool handpiece configured to drive impacting of bone. The second adapter is configured to extend distally from the first adapter. The first adapter includes a first mating feature configured to releasably couple to a second mating element of the second adapter. The first mating feature is one of: a distal-facing cavity having a cross pin extending across a diameter of the cavity, a distally-extending protrusion configured to be seated in a proximal-facing cavity of the surgical implement and having a cross-sectional shape that is one of D-shaped, ovular, Z-shaped, rectangular, star-shaped, hexagonal, or octagonal, and a distal-facing cavity configured to receive therein a proximally-extending protrusion of the surgical implement and having a cross-sectional shape that is one of D-shaped, ovular, Z-shaped, rectangular, star-shaped, hexagonal, or octagonal.

The system can have any number of variations. For example, the second adapter can be non-releasably coupled to a surgical implement configured to impact bone.

For another example, the surgical system can include a surgical implement configured to impact bone, and the second adapter can be configured to releasably couple to the surgical implement.

For yet another example, the surgical system can include a surgical implement configured to impact bone, and the first mating feature of the first adapter can be configured to releasably couple to a selected one of the surgical implement and the second adapter.

For still another example, the first mating feature can be configured to cooperate with the second mating element to prevent rotation of the second adapter relative to the first adapter with the first mating feature releasably coupled to the second mating element.

For another example, the first mating feature can be integrally formed with the first adapter, and the second mating element can be integrally formed with the second adapter.

For still another example, wherein the first mating feature can be configured to releasably attach to the first adapter.

For yet another example, a proximal portion of the first adapter can be configured to be received within the surgical impacting tool handpiece, and the first mating feature can be on a distal-facing surface of the first adapter.

For still another example, the first adapter can be configured to releasably attach to the surgical impacting tool handpiece.

For yet another example, the first adapter can be non-releasably attached to the surgical impacting tool handpiece.

For another example, the surgical system can include the surgical impacting tool handpiece.

In another aspect, a surgical method is provided that in one embodiment includes releasably attaching one of a first adapter or a second adapter to a surgical implement, and, with the one of the first adapter or the second adapter releasably attached to the surgical implement, impacting the surgical implement relative to bone. The first adapter is configured to extend distally from a surgical impacting tool handpiece configured to drive impacting of bone. The first adapter is configured to releasably attach to the surgical impacting tool handpiece, and the first adapter includes a first mating feature configured to releasably couple to a first mating element of a first surgical implement configured to impact bone. The second adapter is configured to extend distally from the surgical impacting tool handpiece. Only one of the first and second adapters is configured to extend distally from the surgical impacting tool handpiece at a time. The second adapter is configured to releasably attach to the surgical impacting tool handpiece, the second adapter includes a second mating feature configured to releasably couple to a second mating element of a second surgical implement configured to impact bone, and the second mating feature is different than the first mating feature.

The method can have any number of variations. For example, the method can also include, before the impacting of the surgical implement, releasably attaching the one of the first adapter or the second adapter to the surgical impacting tool handpiece.

In another embodiment, a surgical method includes releasably attaching an adapter to a surgical implement, and, with the adapter releasably attached to the surgical implement, impacting the surgical implement relative to bone. The adapter is configured to extend distally from a surgical impacting tool handpiece. The adapter includes a mating feature configured to releasably couple to a mating element of the surgical implement configured to impact bone. The mating feature is configured to cooperate with the mating element to prevent rotation of the surgical implement relative to the adapter with the surgical implement releasably attached to the adapter. The mating feature is one of: a distal-facing cavity having a cross pin extending across a diameter of the cavity, a distally-extending protrusion configured to be seated in a proximal-facing cavity of the surgical implement and having a cross-sectional shape that is one of D-shaped, ovular, Z-shaped, rectangular, star-shaped, hexagonal, or octagonal, and a distal-facing cavity configured to receive therein a proximally-extending protrusion of the surgical implement and having a cross-sectional shape that is one of D-shaped, ovular, Z-shaped, rectangular, star-shaped, hexagonal, or octagonal.

The method can vary in any number of ways. For example, the method can include, before the impacting of the surgical implement, releasably attaching the adapter to the surgical impacting tool handpiece.

For another example, the adapter can be non-releasably attached to the surgical impacting tool handpiece.

In another embodiment, a surgical method includes releasably coupling a first mating feature of a first adapter to a second mating element of a second adapter. The method also includes, with the first mating feature of the first adapter releasably coupled to the second mating element of the second adapter and with a surgical implement extending distally from the second adapter, impacting the surgical implement relative to bone. The first adapter is configured to extend distally from a surgical impacting tool handpiece configured to drive impacting of bone. The second adapter is configured to extend distally from the first adapter. The first mating feature is one of: a distal-facing cavity having a cross pin extending across a diameter of the cavity, a distally-extending protrusion configured to be seated in a proximal-facing cavity of the surgical implement and having a cross-sectional shape that is one of D-shaped, ovular, Z-shaped, rectangular, star-shaped, hexagonal, or octagonal, and a distal-facing cavity configured to receive therein a proximally-extending protrusion of the surgical implement and having a cross-sectional shape that is one of D-shaped, ovular, Z-shaped, rectangular, star-shaped, hexagonal, or octagonal.

The method can vary in any number of ways. For example, the second adapter can be non-releasably coupled to the surgical implement.

For another example, the method can include releasably coupling a second mating feature of the second adapter to a third mating element of the surgical implement.

### BRIEF DESCRIPTION OF DRAWINGS

The present invention is described by way of reference to the accompanying figures which are as follows:

FIG. 1 is a side view of an embodiment of a surgical impacting tool handpiece;

FIG. 2 is a top view of the handpiece of FIG. 1;

FIG. 3 is a front view of the handpiece of FIG. 1;

FIG. 4 is a back view of another embodiment of a surgical impacting tool handpiece;

FIG. 5 is a front view of the handpiece of FIG. 4 with one embodiment of an adapter releasably attached thereto;

FIG. 6 is a side view of the handpiece and adapter of FIG. 5;

FIG. 7 is a perspective view of the handpiece and adapter of FIG. 5;

FIG. 8 is another perspective view of the handpiece and adapter of FIG. 5;

FIG. 9 is an exploded view of one embodiment of an adapter;

FIG. 10 is a perspective view of one embodiment of a main body of an adapter;

FIG. 11 is a perspective view of another embodiment of a main body of an adapter;

FIG. 12 is a perspective view of yet another embodiment of a main body of an adapter;

FIG. 13 is an exploded view of another embodiment of an adapter;

FIG. 14 is a perspective view of yet another embodiment of an adapter;

FIG. 15 is a cross-sectional view of the adapter of FIG. 14;

FIG. 16 is a side view of the adapter of FIG. 14;

FIG. 17 is a another side view of the adapter of FIG. 14;

FIG. 18 is a cross-sectional view of the adapter of FIG. 16;

FIG. 19 is a another perspective view of the adapter of FIG. 14;

FIG. 20 is a perspective view of one embodiment of a surgical implement;

FIG. 21 is another perspective view of the surgical implement of FIG. 20; and

FIG. 22 is a perspective view of the adapter of FIG. 9 and another embodiment of an adapter configured to releasably attach to the adapter of FIG. 9.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

A person skilled in the art will recognize that an equivalent to such linear and circular dimensions can easily be determined for any geometric shape. A person skilled in the art will appreciate that a dimension may not be a precise value but nevertheless be considered to be at about that value (for example, may be within +1-0.5% of the value) due to any number of factors such as manufacturing tolerances and sensitivity of measurement equipment. Sizes and shapes of the systems and devices, and the components thereof, can depend at least on the size and shape of components with which the systems and devices will be used.

Various exemplary adapters for surgical impacting tools and methods of using adapters for surgical impacting tools are provided. In general, an adapter can be configured to be releasably attached to a surgical implement configured to impact bone. The adapter can be fixedly, non-releasably attached to a surgical impacting tool handpiece or can be configured to be releasably attached to a surgical impacting tool handpiece. The surgical impacting tool handpiece, such as a handpiece of an orthopedic impactor, is configured to drive impacting of the surgical implement relative to bone. The surgical impacting tool handpiece attached to the adapter is configured to provide a force to the surgical implement, via the adapter, to drive the impacting of the surgical implement. The surgical implement configured to be attached to the adapter can be a broach, chisel, or other surgical implement. Further, each of a plurality of surgical implements configured to be attached to an adapter can be different from one another in one or more aspects, such as shape, size, etc., thereby allowing for a particular surgical implement to be selected by a surgeon (or other medical professional) for optimal desired impacting in a particular surgical procedure being performed on a particular patient's bone.

Different surgical implements and different adapters can have different mating geometries. If the mating geometries of a surgical implement and an adapter do not correspond to each other, the surgical implement and the adapter cannot be coupled together. More particularly, different surgical implements can have different mating elements configured to releasably couple to a mating feature of an adapter. Thus, some surgical implements may only be usable with certain adapters and therefore with certain surgical impacting tool handpieces. Different adapters having different mating features configured to releasably couple to a surgical implement allows a surgical impacting tool handpiece to be releasably coupled to a variety of different surgical implements having different mating elements.

Surgical impacting tool handpieces are typically more expensive than surgical implements, e.g., due to complex designs of surgical impacting tool handpieces and/or surgical impacting tool handpieces including electronic components (e.g., motor, processor, etc.) while surgical implements include no electronic components. Thus, a surgical impacting tool handpiece being selectively attachable to a variety of different surgical implements, via an adapter releasably or non-releasably attached to the surgical impacting tool handpiece, may provide cost effective options for hospitals, medical personnel, etc. since a single, more costly surgical impacting tool handpiece may be purchased for use with a variety of less expensive surgical implements instead of purchasing multiple surgical impacting tool handpieces each being limited for use with certain surgical implements having certain mating elements.

A surgical implement can be a disposable item configured to be used in only one surgical procedure performed on a single patient. In such a scenario, a surgical impacting tool handpiece being selectively attachable to a variety of different surgical implements may allow for the more expensive surgical impacting tool handpiece to be re-used in different surgical procedures performed on different patients.

A surgical implement, whether configured to be disposable or not, may experience wear over time as it impacts bone and need to be disposed of as waste. In such a scenario, a surgical impacting tool handpiece being selectively attachable to a variety of different surgical implements may allow for the more expensive surgical impacting tool handpiece to be re-used even if a surgical implement previously used with the surgical impacting tool handpiece has been disposed of as waste.

In some surgical procedures, a surgeon or other medical personnel may determine that instead of a first surgical implement selected for use with a particular patient, a second surgical implement should be used with the patient. The first and second surgical implements each being configured to releasably couple to the same surgical impacting tool handpiece may allow for one surgical impacting tool handpiece to be available in the operating room (OR) and easily switched from being coupled to the first surgical implement to being coupled to the second surgical implement, thereby saving time during performance of the surgical procedure and/or saving cost by requiring only one surgical impacting tool handpiece to be in the OR.

As mentioned above, an adapter can be configured to be releasably attached to a surgical impacting tool handpiece. The adapter being releasably attachable to the surgical impacting tool handpiece allows the surgical impacting tool handpiece to be releasably attached to a variety of different adapters. Each of the adapters can be different from one another in one or more aspects such as length, mating feature configured to releasably couple to a surgical implement, etc., thereby allowing for a particular adapter to be selected by a surgeon (or other medical professional) for optimal desired impacting in a particular surgical procedure being performed on a particular patient's bone.

As mentioned above, an adapter can be non-releasably attached to a surgical impacting tool handpiece. In some embodiments, the adapter non-releasably attached to a surgical impacting tool handpiece is configured to be releasably attached to a variety of different intermediate adapters. Each of the intermediate adapters is non-releasably attached to a surgical implement or is configured to be releasably attached to a surgical implement. Each of the intermediate adapters can be different from one another in one or more aspects such as length, mating feature configured to releasably couple to a surgical implement, etc., thereby allowing for a particular intermediate adapter to be selected by a surgeon (or other medical professional) for optimal desired impacting in a particular surgical procedure being performed on a particular patient's bone.

In some embodiments, a kit includes one surgical impacting tool handpiece and a plurality of adapters each configured to releasably attach to the surgical impacting tool handpiece and each including a different mating feature. The surgical impacting tool handpiece can therefore be configured to releasably couple to a variety of different surgical implements having different mating elements. Further, the kit can include a plurality of surgical implements each having different mating elements corresponding to the different mating features of the adapter. The resulting tool assembly, which may assembled on demand at a user's choice, can thus include a 3-component chain including the surgical impacting tool handpiece, one of the plurality of adapters, and one of the plurality of surgical implements.

In some embodiments, a kit includes one surgical impacting tool handpiece and a plurality of adapters. One or more of the plurality of adapters (a first number of the plurality of adapters) is configured to releasably couple to the surgical impacting tool handpiece. A remainder of the plurality of adapters (a second number of the plurality of adapters) is configured to releasably couple to at least one of the first number of the plurality of adapters and each includes a different mating feature. The first and second numbers of the plurality of adapters can be the same as one another, e.g., first and second numbers each being 1, 2, 3, 4, 5, or greater than 5. Alternatively, the first and second numbers of the plurality of adapters can be different from one another, e.g., first number being 1 and second number being 2, 3, 4, 5, or greater than 5; first number being 2, 3, 4, 5, or greater than 5 and second number being 1; or first number being 2, 3, 4, 5, or greater than 5 and second number being a different one of 2, 3, 4, 5, or greater than 5. Further, the kit can include a plurality of surgical implements each having different mating elements corresponding to the different mating features of the second number of the plurality of adapters. The resulting tool assembly, which may assembled on demand at a user's choice, can thus include a 4-component chain including the surgical impacting tool handpiece, one of the first number of the plurality of adapters, one of the second number of the plurality of adapters, and one of the plurality of surgical implements.

In some embodiments, a kit includes one surgical impacting tool handpiece and a plurality of adapters. One or more of the plurality of adapters (a first number of the plurality of adapters) is configured to releasably couple to the surgical impacting tool handpiece and each includes a different mating feature. A remainder of the plurality of adapters (a second number of the plurality of adapters) is configured to releasably couple to at least one of the first number of the plurality of adapters, each having different mating elements corresponding to the different mating features of the first number of the plurality of adapters, and each non-releasably attached to a surgical implement. The first and second numbers of the plurality of adapters can be the same as one another, e.g., first and second numbers each being 1, 2, 3, 4, 5, or greater than 5. Alternatively, the first and second numbers of the plurality of adapters can be different from one another, e.g., first number being 1 and second number being 2, 3, 4, 5, or greater than 5; first number being 2, 3, 4, 5, or greater than 5 and second number being 1; or first number being 2, 3, 4, 5, or greater than 5 and second number being a different one of 2, 3, 4, 5, or greater than 5. The resulting tool assembly, which may assembled on demand at a user's choice, can thus include a 3-component chain including the surgical impacting tool handpiece, one of the first number of the plurality of adapters, and one of the second number of the plurality of adapters (which has an integral surgical implement).

In some embodiments, a kit includes one surgical impacting tool handpiece having an adapter non-releasably attached thereto. The kit also includes a plurality of surgical implements each configured to releasably attach to the adapter. Each of the surgical implements has a mating element corresponding to a mating feature of the adapter to allow the surgical implements to be selectively coupled to the adapter. The surgical impacting tool handpiece can therefore be configured to releasably couple to a variety of different surgical implements. The resulting tool assembly, which may assembled on demand at a user's choice, can thus include a 2-component chain including the surgical impacting tool handpiece (which has an integral adapter) and one of the plurality of surgical implements.

In some embodiments, a kit includes one surgical impacting tool handpiece having an adapter non-releasably attached thereto. The kit also includes a plurality of intermediate adapters and a plurality of surgical implements each configured to releasably attach to at least one of the plurality of intermediate adapters. Each of the surgical implements has a mating element corresponding to a mating feature of at least one of the plurality of intermediate adapters to allow the surgical implements to be selectively coupled to the adapter via the intermediate adapter. The surgical impacting tool handpiece can therefore be configured to releasably couple to a variety of different surgical implements. The resulting tool assembly, which may assembled on demand at a user's choice, can thus include a 3-component chain including the surgical impacting tool handpiece (which has an integral adapter), one of the plurality of intermediate adapters, and one of the plurality of surgical implements.

In some embodiments, a tool assembly can include more than two adapters. For example, a tool assembly can include a chain of four or more components including a surgical impacting tool handpiece, at least three adapters, and a surgical implement releasably or non-releasably attached to the at least three adapters, e.g., to a distal-most one of the at least three adapters.

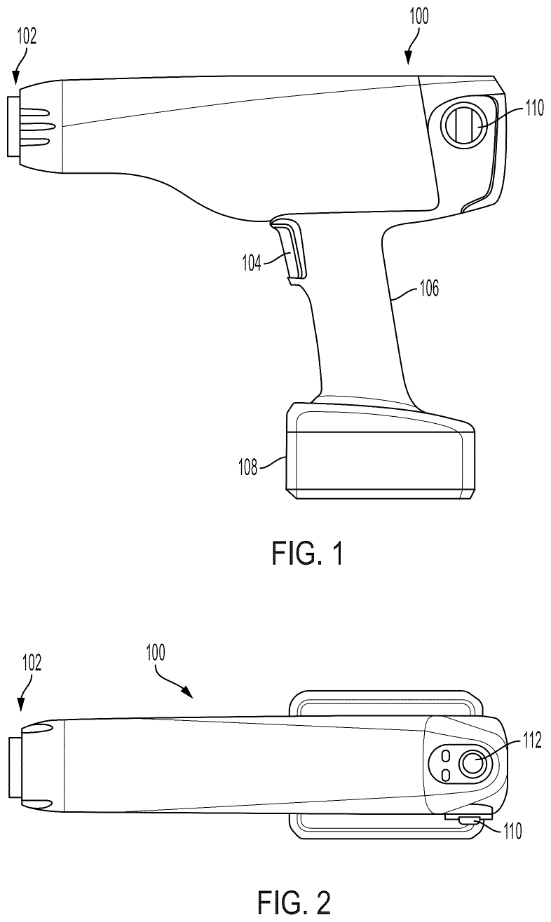

FIGS. 1-3 illustrate one embodiment of a surgical impacting tool handpiece 100. The handpiece is a handpiece of an orthopedic impactor in this illustrated embodiment, but as mentioned above, the surgical impacting tool can be another type of surgical impacting tool. The handpiece 100 includes a locking assembly configured to releasably attach to an adapter. The locking assembly is located at a forward or distal end 102 of the surgical impacting tool handpiece 100 that has a distal-facing cavity 114 formed therein. Various exemplary embodiments of locking assemblies are further described, for example, in U.S. Pat. Pub. No. 2022/0361934 entitled “Surgical Impacting Tool Interfaces” published Nov. 17, 2022 and U.S. Pat. Pub. No. 2024/0156503 entitled “Surgical Impacting Tool Couplings” published May 16, 2024, which are hereby each incorporated by reference in their entirety. Embodiments of the adapter are discussed further below.

The surgical impacting tool handpiece 100 in this illustrated embodiment is configured to releasably attach to an adapter. In other embodiments, the surgical impacting tool handpiece 100 can be non-releasably attached to an adapter.

The adapter, whether releasably attachable to the surgical impacting tool handpiece 100 or non-releasably attached to the surgical impacting tool handpiece 100, can be configured to releasably couple directly to a surgical implement or can be configured to releasably couple indirectly to a surgical implement via one or more intermediate adapters.

The surgical impacting tool handpiece 100 includes an actuator 104 configured to be actuated to drive a surgical implement attached to an adapter that is releasably attached to the handpiece 100 via the locking assembly. The actuator 104 in this illustrated embodiment includes a trigger on a handle 106 of the handpiece 100, but other surgical impacting tools can be actuated in other ways. In an exemplary embodiment, the handpiece 100 is configured to provide forward impacting, in which a forward force is provided by the handpiece 100 for impacting in a forward direction, and rearward impacting, in which a rearward force is provided by the handpiece 100 for impacting in a rearward direction. The forward and rearward impacting can be cyclical with sequential repeated forward and rearward impacts. In some embodiments, the handpiece 100 can be configured to provide only one of forward impacting and rearward impacting.

A power source 108 is configured to releasably attached to the handle 106 of the handpiece 100. The power source 108 includes a battery in this illustrated embodiment, but other power sources are possible. In other embodiments, the handpiece 100 can be releasably attachable to a power source in another way, such as by being plugged into a power source. In still other embodiments, the power source can be non-releasably attached to the handpiece 100, such as by a battery being non-removably disposed in, e.g., the handle 106.

The handpiece 100 includes an energy selector 110 on the handle 106 of the handpiece 100. The energy selector 110 includes a rotary dial in this illustrated embodiment but can have other configurations, such as a lever, a button, etc. The energy selector 110 is configured to allow an energy level to be selected, e.g., high energy or low energy.

The handpiece 100 includes a frequency control 112 on the handle 106 of the handpiece 100. The frequency control 112 includes a button in this illustrated embodiment but can have other configurations, such as a lever, a rotary dial, etc. The frequency control 112 is configured to allow a frequency of impacts to be selected by a user, e.g., slow impacts or fast impacts.

The handpiece 100 can have additional or alternate features. Various exemplary embodiments of surgical impacting tool handpieces including additional or alternate features are further described, for example, in U.S. Pat. Pub. No. 2022/0361934 entitled “Surgical Impacting Tool Interfaces” published Nov. 17, 2022, U.S. Pat. Pub. No. 2013/0161050 entitled “Electric Motor Driven Tool For Orthopedic Impacting” published Jun. 27, 2013, U.S. Pat. No. 10,912,597 entitled “Orthopedic Adapter For An Electric Impacting Tool” issued Feb. 9, 2021, U.S. Pat. No. 11,083,512 entitled “Orthopedic Impacting Device Delivering A Controlled, Repeatable Impact” issued Aug. 10, 2021, U.S. Pat. Pub. No. 11,134,962 entitled “Orthopedic Impacting Device Having A Launched Mass Delivering A Controlled, Repeatable & Reversible Impacting Force” issued Oct. 5, 2021, U.S. Pat. No. 8,393,409 entitled “Electric Motor Driven Tool For Orthopedic Impacting” issued Mar. 12, 2013, U.S. Pat. No. 8,936,105 entitled “Electric Motor Driven Tool For Orthopedic Impacting” issued Jan. 20, 2015, and U.S. Pat. No. 8,695,726 entitled “Electric Motor Driven Tool For Orthopedic Impacting” issued Apr. 15, 2014, which are hereby each incorporated by reference in their entirety.

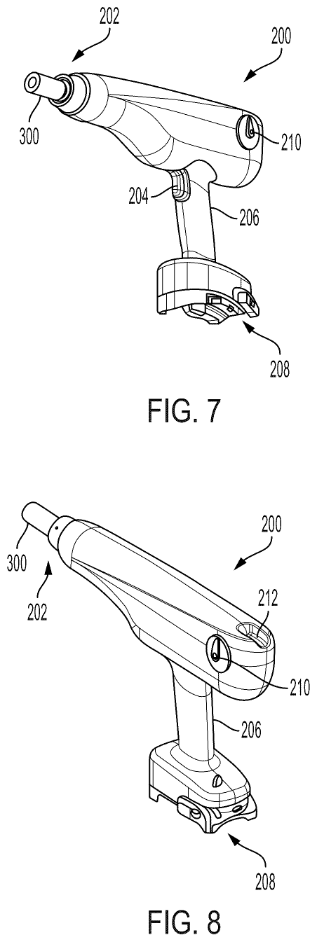

FIGS. 4-8 illustrate another embodiment of a surgical impacting tool handpiece 200 configured to releasably attach to an adapter 300. FIGS. 4-8 show the adapter 300 releasably attached to the handpiece 200 via a locking assembly with a proximal portion of the adapter 300 disposed within a distal-facing cavity (obscured in FIGS. 4-8) formed in the handpiece 200. Embodiments of the adapter 300 are discussed further below. The handpiece 200 is a handpiece of an orthopedic impactor in this illustrated embodiment, but as mentioned above, the surgical impacting tool can be another type of surgical impacting tool. The handpiece 200 is generally configured and used similar to the handpiece 100 of FIG. 1, e.g., includes a locking assembly at a forward or distal end 202 of the handpiece 200 and includes an actuator 204, a handle 206, an energy selector 210 (which is a lever in this illustrated embodiment), and a frequency control 212 (which is a dial in this illustrated embodiment). The handpiece 200 in this illustrated embodiment is configured at a bottom end 208 of the handpiece 200 to releasably attach to a power source, similar to the handpiece 100 being releasably attachable to the power source 108 as discussed above.

The surgical impacting tool handpiece 200 in this illustrated embodiment is configured to releasably attach to the adapter 300. In other embodiments, the surgical impacting tool handpiece 200 can be non-releasably attached to the adapter 300.

The adapter 300, whether releasably attachable to the surgical impacting tool handpiece 200 or non-releasably attached to the surgical impacting tool handpiece 200, can be configured to releasably couple directly to a surgical implement or can be configured to releasably couple indirectly to a surgical implement via one or more intermediate adapters.

The adapter 300, as well as other embodiments of adapters described herein, can be formed from a rigid biocompatible material, such as stainless steel, titanium, or other material. The rigid biocompatible material may allow the adapter to be repeatedly impacted without deforming or breaking over the course of its use with one or more surgical impacting tool handpieces in the performance of one or more surgical procedures.

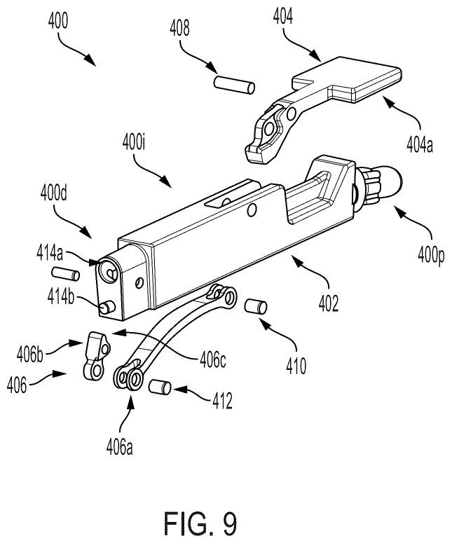

FIG. 9 illustrates another embodiment of an adapter 400 configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. In other embodiments, the adapter 400 can be non-releasably attached to a surgical impacting tool handpiece.

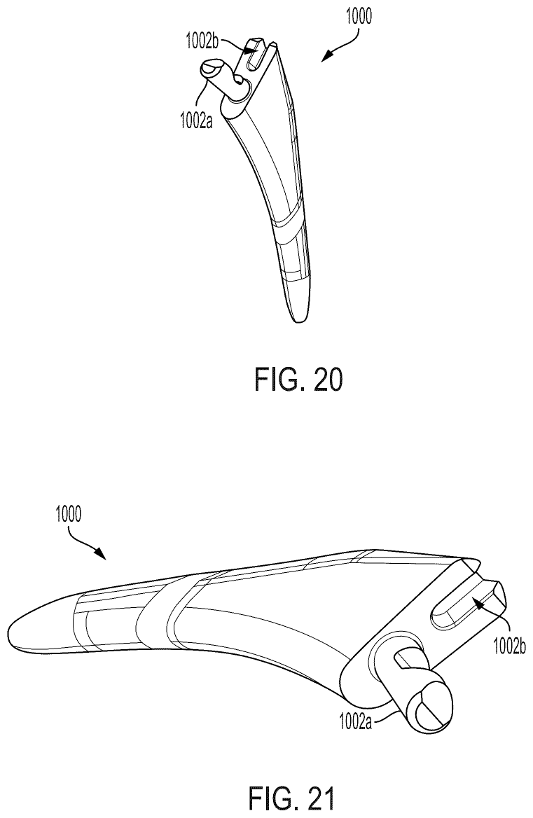

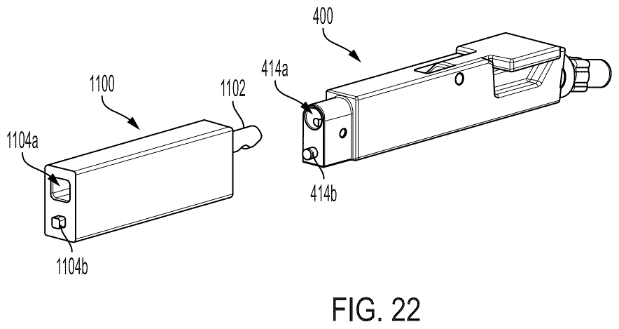

The adapter 400, whether releasably attachable to a surgical impacting tool handpiece or non-releasably attached to a surgical impacting tool handpiece, can be configured to releasably couple directly to a surgical implement or can be configured to releasably couple indirectly to a surgical implement via one or more intermediate adapters. FIGS. 20 and 21 illustrate one embodiment of a surgical implement 1000, discussed further below, configured to releasably attach to the adapter 400 of FIG. 9. FIG. 22 illustrates one embodiment of an intermediate adapter 1100, discussed further below, configured to releasably attach to the adapter 400 of FIG. 9.

The adapter 400 includes a proximal portion 400p, a distal portion 400d, and an intermediate portion 400i located between the proximal and distal portions 400p, 400d. The distal portion 400d of the adapter 400 is configured to releasably attach to a surgical implement or to another adapter configured to releasably attach (directly or indirectly) to a surgical implement. The proximal portion 400p of the adapter 400 is configured to releasably attach to a surgical impacting tool handpiece (or be fixedly attached to a surgical impacting tool handpiece).

The proximal portion 400p of the adapter 400 in this illustrated embodiment has a substantially cylindrical shape and has a substantially circular cross-sectional shape. A person skilled in the art will appreciate that a shape may not be precise, e.g., not precisely cylindrical or precisely circular (for example, be within +1-0.5% of the cylinder and circular diameter), but nevertheless be considered to be substantially that shape due to any number of factors, such as manufacturing tolerances and sensitivity of measurement equipment. The substantially cylindrical shape and substantially circular cross-sectional shape of the proximal portion 400p may reduce angular play of the adapter 400 with respect to a surgical impacting tool handpiece within which the proximal portion 400p is at least partially disposed, e.g., disposed within a distal-facing cavity formed in the handpiece. Less angular play between an adapter and a surgical impacting tool handpiece within which the adapter is partially disposed may allow for a more secure fit between the adapter and handpiece and/or may facilitate impacting more precisely along the adapter's longitudinal axis, and thus provide for more predictably directed impacting on bone, since the adapter's angular movement relative to the longitudinal axis is reduced. In other embodiments, the proximal portion 400p of the adapter 400 may have a different shape and cross-sectional shape. Similarly, the substantially cylindrical shape and substantially circular cross-sectional shape of the proximal portion 400p may reduce angular play of the adapter 400 with respect to an intermediate adapter within which the proximal portion 400p is at least partially disposed, e.g., disposed within a distal-facing cavity formed in the intermediate adapter.

The adapter 400 in this illustrated embodiment includes a main body 402, a proximal pivotable arm 404, and a distal pivotable arm 406. In some embodiments, the proximal pivotable arm 404 and/or the distal pivotable arm 406 can be omitted.

A first pivot pin 408 pivotably connects the proximal pivotable arm 404 to the main body 402. The proximal pivotable arm 404 is configured to pivot relative to the main body 402 at the first pivot pin 408. The proximal pivotable arm 404 is configured to dynamically pivot relative to the main body 402 during connection of the adapter 400 with a surgical impacting tool handpiece, e.g., during insertion of the proximal portion 400p into a surgical impacting tool handpiece. A surface 404a of the proximal pivotable arm 404 is configured to contact a surface of the surgical impacting tool handpiece to help stabilize the adapter 400 relative to the surgical impacting tool handpiece.

A second pivot pin 410 pivotably connects the distal pivotable arm 406 to the main body 402. The distal pivotable arm 406 includes an elongate portion 406a and a catch component 406b pivotably coupled to the elongate portion 406a with a third pivot pin 412. The distal pivotable arm 406 is configured to pivot relative to the main body 402 at the second pivot pin 410. The catch component 406b of the distal pivotable arm 406 is configured to pivot relative to the elongate portion 406a of the distal pivotable arm 406 at the third pivot pin 412. The elongate portion 406a of the distal pivotable arm 406 is configured to dynamically pivot relative to the main body 402 and the catch component 406b of the distal pivotable arm 406 is configured to dynamically pivot relative to the elongate portion 406a of the distal pivotable arm 406 during connection of the adapter 400 with a surgical implement or an intermediate adapter. A surface 406c of the catch component 406b is configured to contact a surface of the surgical implement or the intermediate adapter to help stabilize the adapter 400 relative to the surgical implement or the intermediate adapter.

The catch component's surface 406c can include a retention feature configured to improve retention of the surgical implement to the adapter 400. Various retention features are possible. For example, in some embodiments, the retention feature can include a textured surface, e.g., raised bumps, cross-hatching, etc. For another example, in other embodiments, the retention feature can include at least one magnet configured to magnetically attract to the surgical implement.

Catch components of other embodiments of adapters discussed herein that include a catch component can include a retention feature.

As mentioned above, the distal portion 400d of the adapter 400 is configured to releasably couple to a surgical implement or to an intermediate adapter. The adapter 400 can thus be coupled (directly or indirectly) to any of a variety of surgical implements, thereby increasing versatility of the adapter 400.

The distal portion 400d of the adapter 400 in this illustrated embodiment has a rectangular-dome cross-sectional shape and a shape similar to a traditional mailbox. In other embodiments, the distal portion 400d of the adapter 400 may have a different shape and cross-sectional shape.

The distal portion 400d of the adapter 400 in this illustrated embodiment is an integral part of the main body 402. In other embodiments, the distal portion 400d can be an independent component configured to be releasably attached to a remainder of the main body 402. The distal portion 400d being releasably attachable may allow the adapter 400 to be used with existing surgical impacting tool handpieces that would otherwise not be able to be used with surgical implements having a mating element configured to releasably mate with the distal portion's mating feature.

Other embodiments of adapters discussed herein can include a distal portion that is non-releasable from a main body or that is configured to releasably couple to a main body.

The adapter 400 includes a mating feature in the distal portion 400d that is configured to mate with a mating element of a surgical implement or an intermediate adapter. The mating feature is located on a distal face of the adapter 400 and is thus distal-facing. The mating feature in this illustrated embodiment includes a cavity 414a formed in the adapter 400 (the main body 402 thereof) and a protrusion (also referred to herein as a “boss”) 414b formed on the adapter 400 (the main body 402 thereof) and extending distally. The cavity 414a in this illustrated embodiment has a substantially cylindrical shape and has a substantially circular cross-sectional shape. A person skilled in the art will appreciate that a shape may not be precise, e.g., not precisely cylindrical or precisely circular (for example, be within +1-0.5% of the cylinder and circular diameter), but nevertheless be considered to be substantially that shape due to any number of factors, such as manufacturing tolerances and sensitivity of measurement equipment. Other shapes of the cavity 414a are possible, e.g., box shaped, pyramid shaped, half-sphere shaped, etc. The protrusion 414b in this illustrated embodiment has a substantially cylindrical shape and has a substantially circular cross-sectional shape. Other shapes of the protrusion 414b are possible, e.g., box shaped, pyramid shaped, half-sphere shaped, etc.

The mating feature is configured to mate with a mating element of a surgical implement or an intermediate adapter that has a geometry corresponding to the mating feature's geometry. In this way, only a surgical implement or an intermediate adapter having mating element geometry corresponding to the geometry of the adapter's mating feature can be mated to the adapter. In this illustrated embodiment, the mating feature cavity 414a is configured to receive therein a corresponding proximally-extending protrusion of a surgical implement or an intermediate adapter, namely a protrusion having a substantially cylindrical shape and a substantially circular cross-sectional shape, and the mating feature boss 414b is configured to be received in a corresponding distally-facing cavity of the surgical implement or the intermediate adapter, namely a cavity having a substantially cylindrical shape and a substantially circular cross-sectional shape.

As in this illustrated embodiment, the mating feature of the adapter 400 can be configured as an anti-rotation feature configured to prevent rotation of a surgical implement or an intermediate adapter relative to the adapter 400 with the surgical implement or the intermediate adapter mated to the adapter 400. Preventing rotation of a surgical implement or an intermediate adapter relative to the adapter 400 may provide for stable, predictably-placed impacting. In this illustrated embodiment, the mating feature cavity 414a and the mating feature protrusion 414b are configured to cooperate to prevent rotation of a surgical implement or an intermediate adapter relative to the adapter 400 because with the surgical implement or the intermediate adapter mated to both of the mating feature cavity 414a and the mating feature protrusion 414b, the surgical implement or the intermediate adapter cannot rotate relative to the adapter 400.

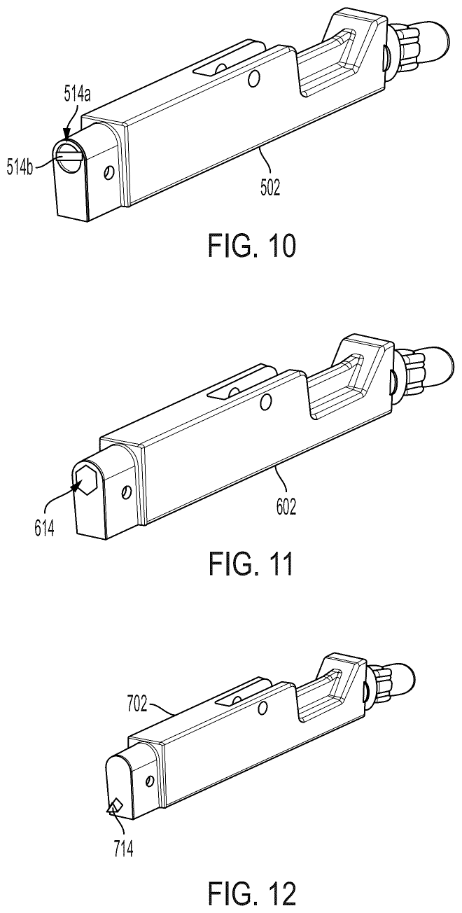

FIG. 10 illustrates another embodiment of an adapter configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter of FIG. 10 is configured and used similar to that discussed above with respect to the adapter 400 of FIG. 9, e.g., includes a proximal portion, a distal portion, an intermediate portion located between the proximal and distal portions, a main body 502, a proximal pivotable arm (not shown), and a distal pivotable arm (not shown). In some embodiments, the proximal pivotable arm and/or the distal pivotable arm can be omitted.

The adapter main body 502 of FIG. 10 is configured and used similar to that discussed above with respect to the adapter main body 402 of FIG. 9 except for the mating feature. A mating feature of FIG. 10 is located on a distal face of the adapter and is thus distal-facing, but in the illustrated embodiment of FIG. 10, the mating feature includes a cavity 514a formed in the adapter (the main body 502 thereof) and having a cross pin 514b extending across a diameter of the cavity 514a. The cavity 514a in this illustrated embodiment has a substantially cylindrical shape and has a substantially circular cross-sectional shape, but other shapes are possible. The cross pin 514b in this illustrated embodiment has a substantially cylindrical shape and has a substantially circular cross-sectional shape, but other shapes are possible.

The mating feature is configured to mate with a mating element of a surgical implement (e.g., the surgical implement 1000 of FIGS. 20 and 21 or other surgical implement) or an intermediate adapter (e.g., the intermediate adapter 1100 of FIG. 22 or other adapter) that has a geometry corresponding to the mating feature's geometry. In this way, only a surgical implement or an intermediate adapter having mating element geometry corresponding to the geometry of the adapter's mating feature can be mated to the adapter. In this illustrated embodiment, the mating feature cavity 514a is configured to receive therein a corresponding proximally-extending protrusion of a surgical implement or an intermediate adapter. The proximally-extending protrusion can include a pair of protrusions configured to be received in the cavity 514a with a first one of the protrusions received on a first side of the cross pin 514b in a first half of the cavity 514a and a second one of the protrusions received on a second side of the cross pin 514b in a second half of the cavity 514a.

The mating feature of the adapter of FIG. 10 is configured as an anti-rotation feature configured to prevent rotation of a surgical implement or an intermediate adapter relative to the adapter with the surgical implement or the intermediate adapter mated to the adapter. The mating feature cavity 514a alone could not prevent rotation of the surgical implement or the intermediate adapter relative to the adapter because of its cylindrical shape. The mating feature cross pin 514b is configured to prevent rotation of a surgical implement or an intermediate adapter relative to the adapter because with the surgical implement or the intermediate adapter mated to both of the mating feature cavity 514a and the mating feature cross pin 514b, the surgical implement or the intermediate adapter cannot rotate relative to the adapter.

In this illustrated embodiment, the distal-facing surface of the adapter in which the mating feature cavity 514a is formed is free of any protrusion extending distally therefrom and is free any other cavities formed therein. The mating feature of FIG. 10 is thus a singular mating feature, which may simplify manufacturing and/or simplify releasable coupling of a surgical implement or an intermediate adapter to the adapter, as compared to a mating feature having multiple elements on a distal-facing surface such as the mating feature of FIG. 9 that includes the cavity 414a formed in a distal-facing surface and the protrusion 414b extending from the distal-facing surface.

The adapter in this illustrated embodiment of FIG. 10 is configured to releasably attach to a surgical impacting tool handpiece. In other embodiments, the adapter can be non-releasably attached to a surgical impacting tool handpiece.

FIG. 11 illustrates another embodiment of an adapter configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter of FIG. 11 is configured and used similar to that discussed above with respect to the adapter 400 of FIG. 9, e.g., includes a proximal portion, a distal portion, an intermediate portion located between the proximal and distal portions, a main body 602, a proximal pivotable arm (not shown), and a distal pivotable arm (not shown). In some embodiments, the proximal pivotable arm and/or the distal pivotable arm can be omitted.

The adapter main body 602 of FIG. 11 is configured and used similar to that discussed above with respect to the adapter main body 402 of FIG. 9 except for the mating feature. A mating feature 614 of FIG. 11 is located on a distal face of the adapter and is thus distal-facing, but in the illustrated embodiment of FIG. 11, the mating feature 614 includes a cavity formed in the adapter (the main body 602 thereof). The cavity 614 in this illustrated embodiment has a substantially hexagonal shape and has a substantially hexagonal cross-sectional shape.

The mating feature is configured to mate with a mating element of a surgical implement (e.g., the surgical implement 1000 of FIGS. 20 and 21 or other surgical implement) or an intermediate adapter (e.g., the intermediate adapter 1100 of FIG. 22 or other adapter) that has a geometry corresponding to the mating feature's geometry. In this way, only a surgical implement or an intermediate adapter having mating element geometry corresponding to the geometry of the adapter's mating feature can be mated to the adapter. In this illustrated embodiment, the mating feature cavity 614 is configured to receive therein a corresponding proximally-extending protrusion of a surgical implement or an intermediate adapter that has a substantially hexagonal cross-sectional shape.

The mating feature 614 of the adapter of FIG. 11 is configured as an anti-rotation feature configured to prevent rotation of a surgical implement or an intermediate adapter relative to the adapter with the surgical implement or the intermediate adapter mated to the adapter. Unlike the mating feature cavities 414a, 514a of FIGS. 9 and 10, the mating feature cavity 614 alone is configured to prevent rotation of the surgical implement or the intermediate adapter relative to the adapter because of its hexagonal shape.

In this illustrated embodiment, the distal-facing surface of the adapter in which the mating feature cavity 614 is formed is free of any protrusion extending distally therefrom and is free any other cavities formed therein. The mating feature 614 of FIG. 11 is thus a singular mating feature, which may simplify manufacturing and/or simplify releasable coupling of a surgical implement or an intermediate adapter to the adapter, as compared to a mating feature having multiple elements on a distal-facing surface such as the mating feature of FIG. 9 that includes the cavity 414a formed in a distal-facing surface and the protrusion 414b extending from the distal-facing surface.

The adapter of FIG. 11 is configured to be coupled to a surgical implement or an intermediate adapter at a plurality of predetermined angular orientations. A surgical implement or an intermediate adapter coupled to the adapter, and thus operatively coupled to the surgical impacting tool handpiece via the adapter, can therefore be attached to the surgical impacting tool handpiece at a plurality of predetermined angular orientations relative to the surgical impacting tool handpiece. Depending on one or more factors such as surgeon preference, which hand (left or right) of a user is holding the surgical impacting tool, which bone of a patient the surgical implement will be impacting, and a position of a patient relative to a user of the surgical impacting tool, a certain angular orientation of the surgical implement or the intermediate adapter may be more desirable than another angular orientation of the surgical implement or the intermediate adapter.

A number of sides defining the shape of the mating feature cavity 614, and in particular planar surfaces thereof, defines a number of the predetermined angular orientations. The adapter in the illustrated embodiment is thus configured to be releasably attached to a surgical implement or an intermediate adapter at six predetermined angular orientations relative to the handpiece: about zero degrees, about sixty degrees, about one hundred twenty degrees, about one hundred eighty degrees, and about two hundred forty degrees. Thus, in this illustrated embodiment, each of the predetermined angular orientations is about sixty degrees apart from one another.

Other examples of the shape of the mating feature cavity 614 configured as an anti-rotation feature and that defines a number of a plurality of predetermined angular orientations at which a surgical implement or an intermediate adapter can be releasably coupled to the adapter include N-sided polygons, where N is an integer two or greater, e.g., an equilateral triangle, a square, a rectangle, a pentagon, an octagon, a star shape, a Z-shape, a D-shape, an hourglass shape, etc. Another example of the shape of the mating feature cavity 614 configured as an anti-rotation feature and that defines a number of a plurality of predetermined angular orientations at which a surgical implement or an intermediate adapter can be releasably coupled to the adapter includes an oval shape, which defines two predetermined angular orientations.

In some embodiments, instead of the mating feature cavity 614 defining a plural number of predetermined angular orientations at which a surgical implement or an intermediate adapter can be releasably coupled to the adapter, the mating feature cavity 614 can define a single predetermined angular orientation at which a surgical implement or an intermediate adapter can be releasably coupled to the adapter. Examples of the shape of the mating feature cavity 614 configured as an anti-rotation feature and that defines a single predetermined angular orientation at which a surgical implement or an intermediate adapter can be releasably coupled to the adapter include an isosceles triangle, an irregular polygon shape (e.g., a pear shape, a non-convex pentagon, etc.), etc.

The adapter in this illustrated embodiment of FIG. 11 is configured to releasably attach to a surgical impacting tool handpiece. In other embodiments, the adapter can be non-releasably attached to a surgical impacting tool handpiece.

FIG. 12 illustrates another embodiment of an adapter configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter of FIG. 12 is configured and used similar to that discussed above with respect to the adapter 400 of FIG. 9, e.g., includes a proximal portion, a distal portion, an intermediate portion located between the proximal and distal portions, a main body 702, a proximal pivotable arm (not shown), and a distal pivotable arm (not shown). In some embodiments, the proximal pivotable arm and/or the distal pivotable arm can be omitted.

The adapter main body 702 of FIG. 12 is configured and used similar to that discussed above with respect to the adapter main body 402 of FIG. 9 except for the mating feature. A mating feature 714 of FIG. 12 is located on a distal face of the adapter and is thus distal-facing, but in the illustrated embodiment of FIG. 12, the mating feature 714 includes a boss formed on the adapter (the main body 702 thereof). The boss 714 in this illustrated embodiment has a substantially triangular-prism shape and has a substantially equilateral triangular cross-sectional shape.

The mating feature is configured to mate with a mating element of a surgical implement (e.g., the surgical implement 1000 of FIGS. 20 and 21 or other surgical implement) or an intermediate adapter (e.g., the intermediate adapter 1100 of FIG. 22 or other adapter) that has a geometry corresponding to the mating feature's geometry. In this way, only a surgical implement or an intermediate adapter having mating element geometry corresponding to the geometry of the adapter's mating feature can be mated to the adapter. In this illustrated embodiment, the mating feature protrusion 714 is configured to be received in a corresponding cavity of a surgical implement or an intermediate adapter that has a substantially triangular-prism shape and a substantially triangular cross-sectional shape.

The mating feature 714 of the adapter of FIG. 12 is configured as an anti-rotation feature configured to prevent rotation of a surgical implement or an intermediate adapter relative to the adapter with the surgical implement or the intermediate adapter mated to the adapter. Unlike the mating feature protrusion 414b of FIG. 9, the mating feature protrusion 714 alone is configured to prevent rotation of the surgical implement or the intermediate adapter relative to the adapter because of its triangular shape.

In this illustrated embodiment, the distal-facing surface of the adapter on which the mating feature protrusion 714 is formed is free of any other protrusion extending distally therefrom and is free any cavities formed therein. The mating feature 714 of FIG. 12 is thus a singular mating feature.

The adapter of FIG. 12 is configured to be coupled to a surgical implement or an intermediate adapter at a plurality of predetermined angular orientations. A surgical implement or an intermediate adapter coupled to the adapter, and thus operatively coupled to the surgical impacting tool handpiece via the adapter, can therefore be attached to the surgical impacting tool handpiece at a plurality of predetermined angular orientations relative to the surgical impacting tool handpiece. A number of sides defining the shape of the mating feature protrusion 714, and in particular planar surfaces thereof, defines a number of the predetermined angular orientations. The adapter in the illustrated embodiment is thus configured to be releasably attached to a surgical implement at three predetermined angular orientations relative to the handpiece: about zero degrees, about one hundred twenty degrees, and about two hundred forty degrees. Thus, in this illustrated embodiment, each of the predetermined angular orientations is about one hundred twenty degrees apart from one another.

Other examples of the shape of the mating feature protrusion 714 configured as an anti-rotation feature and that defines a number of a plurality of predetermined angular orientations at which a surgical implement can be releasably coupled to the adapter include N-sided polygons, where N is an integer two or greater, e.g., a square, a rectangle, a pentagon, an octagon, a star shape, a Z-shape, a D-shape, an hourglass shape, etc. Another example of the shape of the mating feature protrusion 714 configured as an anti-rotation feature and that defines a number of a plurality of predetermined angular orientations at which a surgical implement or an intermediate adapter can be releasably coupled to the adapter includes an oval shape, which defines two predetermined angular orientations.

In some embodiments, instead of the mating feature protrusion 714 defining a plural number of predetermined angular orientations at which a surgical implement or an intermediate adapter can be releasably coupled to the adapter, the mating feature protrusion 714 can define a single predetermined angular orientation at which a surgical implement or an intermediate adapter can be releasably coupled to the adapter. Examples of the shape of the mating feature protrusion 714 configured as an anti-rotation feature and that defines a single predetermined angular orientation at which a surgical implement or an intermediate adapter can be releasably coupled to the adapter include an isosceles triangle, an irregular polygon shape (e.g., a pear shape, a non-convex pentagon, etc.), etc.

The adapter in this illustrated embodiment of FIG. 12 is configured to releasably attach to a surgical impacting tool handpiece. In other embodiments, the adapter can be non-releasably attached to a surgical impacting tool handpiece.

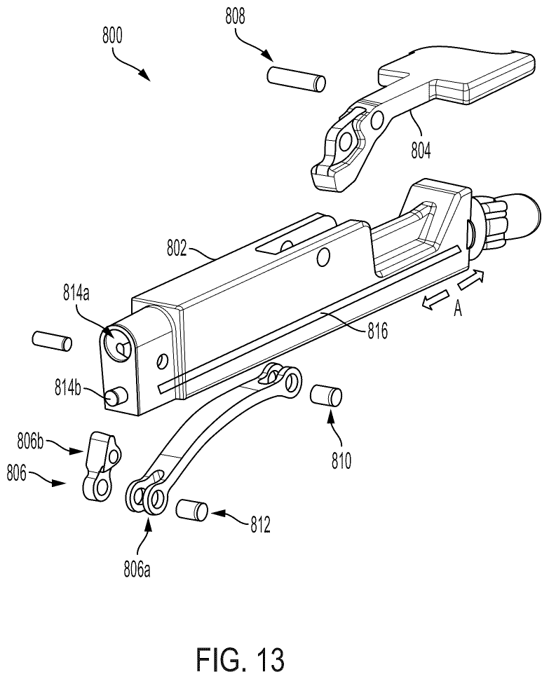

FIG. 13 illustrates another embodiment of an adapter 800 configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter 800 of FIG. 13 is configured and used similar to that discussed above with respect to the adapter 400 of FIG. 9, e.g., includes a proximal portion, a distal portion, an intermediate portion located between the proximal and distal portions, a main body 802, a proximal pivotable arm 804 (partially shown in FIG. 13) pivotally connected to the main body 802 with a first pivot pin 808, a distal pivotable arm (including an elongate portion 806a and a catch component 806b pivotably coupled to the elongate portion 806a with a third pivot pin 812) pivotally connected to the main body 802 with a second pivot pin 810, and a mating feature including a cavity 814a formed in the adapter 800 (the main body 802 thereof) and a protrusion 814b formed on the adapter 800 (the main body 802 thereof) and extending distally. In some embodiments, the proximal pivotable arm and/or the distal pivotable arm can be omitted.

The adapter 800 of FIG. 12 is configured and used similar to that discussed above with respect to the adapter 400 of FIG. 9 except for the distal pivotable arm. In this illustrated embodiment, the catch component 806b of the distal pivotable arm 806 is not configured to dynamically pivot relative to the elongate portion 806a of the distal pivotable arm 806 during connection of the adapter 800 with a surgical implement or an intermediate adapter or at another time. Instead, the pivoting of the catch component 806b of the distal pivotable arm 806 relative to the elongate portion 806a of the distal pivotable arm 806 is configured to be manually controlled. The catch component's pivoting being manually controllable may allow for a more secure retention of the surgical implement coupled to the adapter 800.

The adapter 800 includes a drive mechanism 816 configured to drive manual pivoting of the catch component 806b. The drive mechanism 816 in this illustrated embodiment includes a threaded rod fixedly attached to the catch component 806b and threadably mated with a thread of the main body 802. Manually rotating the drive mechanism 816, either directly by hand or indirectly by using a separate tool, is configured to longitudinally translate the drive mechanism 816 proximally or distally, as shown by arrows A. Whether the drive mechanism 816 moves proximally or distally depends on a direction of the drive mechanism's rotation relative to the main body 802. The longitudinal translation of the drive mechanism 816 is configured to pivot the catch component 806b relative to the elongate portion 806a of the distal pivotable arm 806, pivoting away from or distally in response to the drive mechanism 816 moving distally and pivoting toward or proximally in response to the drive mechanism 816 moving proximally.

Catch components of other embodiments of adapters discussed herein that include a catch component can be configured to be manually controlled, e.g., using a drive mechanism, instead of being configured to dynamically pivot.

The adapter 800 in this illustrated embodiment is configured to releasably attach to a surgical impacting tool handpiece. In other embodiments, the adapter 800 can be non-releasably attached to a surgical impacting tool handpiece.

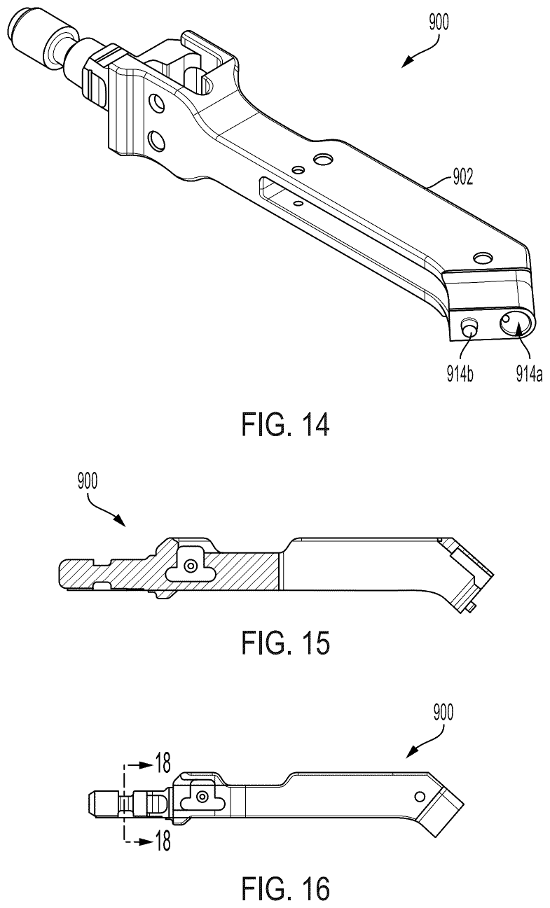

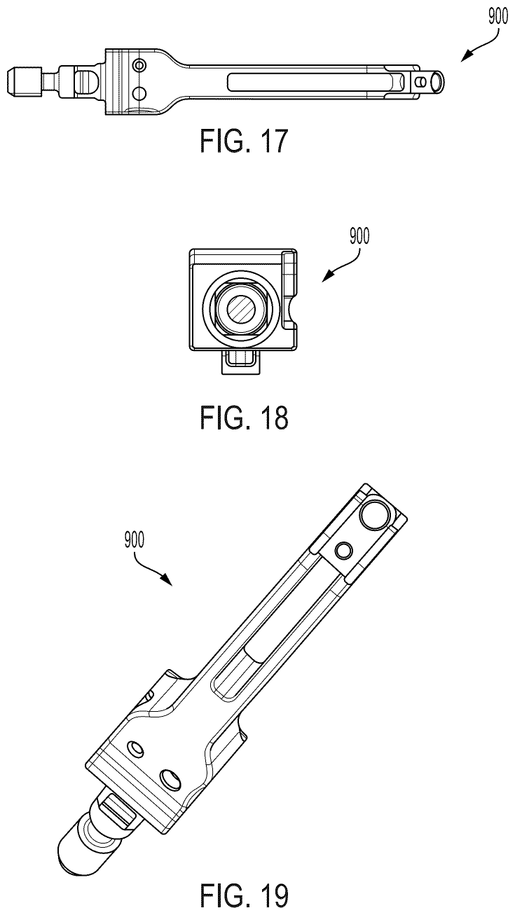

FIGS. 14-19 illustrate another embodiment of an adapter 900 configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter 900 of FIGS. 14-19 is configured and used similar to that discussed above with respect to the adapter 400 of FIG. 9, e.g., includes a proximal portion, a distal portion, an intermediate portion located between the proximal and distal portions, a main body 902, and a mating feature including a cavity 914a formed in the adapter 900 (the main body 902 thereof) and a protrusion 914b formed on the adapter 900 (the main body 902 thereof) and extending distally. However, the adapter 900 differs from the adapter 400 of FIG. 9 in that the adapter 900 does not include proximal or distal pivotable arms and in that the distal portion is enlarged and includes a curved portion at a distal end of the adapter 900. The adapter 900 is configured for use in impacting a hip bone. The curved portion of the adapter 900 may help a surgical implement releasably coupled (directly or indirectly) to the distal end of the adapter 900 access the hip bone.

Other embodiments of adapters discussed herein can include a distal portion that is enlarged and includes a curved portion at a distal end of the adapter similar to the adapter 900.

The adapter 900 in this illustrated embodiment is configured to releasably attach to a surgical impacting tool handpiece. In other embodiments, the adapter 900 can be non-releasably attached to a surgical impacting tool handpiece.

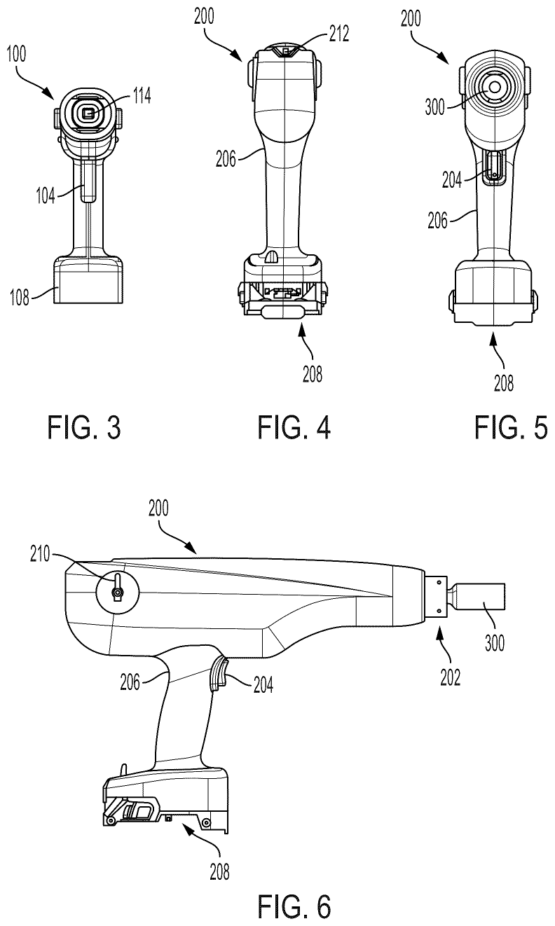

As mentioned above, various surgical implements such as chisels and broaches can be configured to releasably attach to an adapter, such as the adapter 300 of FIGS. 3 and 5-8, the adapter 400 of FIG. 9, the adapter 1100 of FIG. 22, the adapter of FIG. 10, the adapter of FIG. 11, the adapter of FIG. 12, the adapter 800 of FIG. 13, the adapter 900 of FIGS. 14-19, or another adapter. FIGS. 20 and 21 illustrate one embodiment of a surgical implement 1000 configured to releasably attach to the adapter. The surgical implement 1000 in this illustrated embodiment is a hip broach configured for impacting a hip bone. The surgical implement 1000 includes a mating element configured to releasably mate with a mating feature of an adapter. The mating element in this illustrated embodiment includes a protrusion 1002a and a cavity 1002b. The protrusion 1002a extends proximally and is configured to be received in a corresponding mating feature cavity of an adapter. The protrusion 1002a has a varying cross-sectional shape along its length, but other shapes are possible similar to that discussed above regarding mating features. The cavity 1002b is formed in a proximal face of the surgical implement 1000 and is configured to receive therein a corresponding mating feature protrusion of the adapter. The cavity 1002b has a U-shaped cross-sectional shape, but other shapes are possible similar to that discussed above regarding mating features.

As mentioned above, instead of a surgical implement being releasably attached to an adapter, such as the adapter 300 of FIGS. 3 and 5-8, the adapter 400 of FIG. 9, the adapter 1100 of FIG. 22, the adapter of FIG. 10, the adapter of FIG. 11, the adapter of FIG. 12, the adapter 800 of FIG. 13, the adapter 900 of FIGS. 14-19, or another adapter, by releasably mating with a mating feature of the adapter to extend distally from the adapter, a second adapter can be releasably attached to the adapter by releasably mating with the mating feature of the adapter to extend distally from the adapter. The second adapter may provide increased versatility of the adapter to which the second adapter is coupled. As also mentioned above, a chain of adapters coupled together can include two adapters or can include more than two adapters, e.g., three, four, etc.

FIG. 22 illustrates one embodiment of a second adapter 1100 configured to releasably attach to another adapter, referred to herein for ease of explanation as a “first adapter.” The adapter 1100 of FIG. 22 is configured and used similar to that discussed above with respect to the adapter 400 of FIG. 9, e.g., includes a proximal portion, a distal portion, an intermediate portion located between the proximal and distal portions, and a main body. The adapter 1100 of FIG. 22 does not include a proximal pivotable arm or a distal pivotable arm. However, in other embodiments, a second adapter can include a proximal pivotable arm and/or a distal pivotable arm.

The second adapter 1100 includes a mating element configured to releasably mate with a mating feature of the first adapter. FIG. 22 illustrates the second adapter 1100 being configured to releasably attach to the adapter 400 of FIG. 9 as the first adapter. However, other second adapters can be configured and used similar to the second adapter 1100 to releasably attach to other embodiments of adapters described herein, e.g., by having a mating element appropriate for coupling with a particular adapter's mating feature. Additionally, the second adapter 1100 can releasably attach to a first adapter other than the adapter 400 of FIG. 9.

The mating element of the second adapter 1100 in this illustrated embodiment includes a protrusion 1102 and a cavity (obscured in FIG. 22). The protrusion 1102 extends proximally and is configured to be received in a corresponding mating feature cavity of an adapter, which in this illustrated embodiment is the cavity 414a of the first adapter 400. The cavity is formed in a proximal face of the second adapter 1100 and is configured to receive therein a corresponding mating feature protrusion of the adapter, which in this illustrated embodiment is the protrusion 414b of the first adapter 400. The mating element protrusion 1102 of the second adapter 1100 in this illustrated embodiment thus has a substantially cylindrical shape and has a substantially circular cross-sectional shape but, as discussed herein, other shapes and cross-sectional shapes are possible. The mating element cavity of the second adapter 1100 in this illustrated embodiment thus has a substantially cylindrical shape and has a substantially circular cross-sectional shape but, as discussed herein, other shapes and cross-sectional shapes are possible.

The second adapter 1100 includes a mating feature configured to releasably mate with a mating element of a surgical implement or with a mating element of another (third) adapter. The mating feature of the second adapter 1100 is located on a distal face of the second adapter 1100 and is thus distal-facing. In the illustrated embodiment of FIG. 22, the mating feature includes a cavity 1104a formed in the second adapter 1100 (the main body thereof) and a protrusion 1104b extending distally from the distal face of the second adapter 1100. The mating feature cavity 1104a in this illustrated embodiment has a substantially rectangular shape and has a substantially rectangular cross-sectional shape, but, as discussed herein, other shapes and cross-sectional shapes are possible. The mating feature protrusion 1104b in this illustrated embodiment has a substantially rectangular shape and has a substantially rectangular cross-sectional shape, but, as discussed herein, other shapes and cross-sectional shapes are possible. Additionally, the mating feature cavity 1104a is located above the mating feature protrusion 1104b in this illustrated embodiment, but the mating feature cavity 1104a and the mating feature protrusion 1104b can be otherwise arranged.

The mating feature of the second adapter 1100 is configured to mate with a mating element of a surgical implement or an intermediate adapter that has a geometry corresponding to the mating feature's geometry. In this way, only a surgical implement or an intermediate adapter having mating element geometry corresponding to the geometry of the adapter's mating feature can be mated to the second adapter 1100. In this illustrated embodiment, the mating feature cavity 1104a is configured to receive therein a corresponding proximally-extending protrusion of a surgical implement or an intermediate adapter that has a substantially rectangular cross-sectional shape and the mating feature protrusion 1104b is configured to be received in a corresponding proximal cavity of a surgical implement or an intermediate adapter that has a substantially rectangular cross-sectional shape.

The mating feature of the second adapter 1100 of FIG. 22 is configured as an anti-rotation feature configured to prevent rotation of a surgical implement or an intermediate adapter relative to the second adapter 1100 with the surgical implement or the intermediate adapter mated to the second adapter 1100. In this illustrated embodiment, the mating feature cavity 1104a and the mating feature protrusion 1104b are configured to cooperate to prevent rotation of a surgical implement or an intermediate adapter relative to the second adapter 1100 because with the surgical implement or the intermediate adapter mated to both of the mating feature cavity 1104a and the mating feature protrusion 1104b, the surgical implement or the intermediate adapter cannot rotate relative to the second adapter 1100.

The second adapter 1100 in this illustrated embodiment of FIG. 22 is configured to releasably attach to a surgical implement or to another intermediate adapter. In other embodiments, the second adapter 1100 can be non-releasably attached to a surgical implement. In such embodiments, the second adapter 1100 cannot be releasably attached to another intermediate adapter.

The devices disclosed herein can be designed to be disposed of after a single use, or they can be designed to be used multiple times. In either case, however, a device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices described herein can be processed before use. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation or toxic gas that can penetrate the container, such as Ethylene Oxide, gamma radiation, x-rays, or high energy electrons. The radiation kills bacteria on the instrument and in the container. The sterilized instrument can then be stored in the sterile container. The sealed container keeps the instrument sterile until it is opened in a medical facility.

Sterilization can be done by any number of ways known to those skilled in the art including beta or gamma radiation, ethylene oxide, steam, and a liquid bath (e.g., cold soak).

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

1-26. (canceled)

27. A surgical system, comprising:
a surgical impacting tool handpiece configured to drive impacting of bone; and
an adapter configured to extend distally from the surgical impacting tool handpiece, wherein the adapter includes a mating feature configured to releasably couple to a mating element of a surgical implement configured to impact bone, the mating feature is configured to cooperate with the mating element to prevent rotation of the surgical implement relative to the adapter with the surgical implement releasably attached to the adapter, and the mating feature is one of:
a distal-facing cavity having a cross pin extending across a diameter of the cavity,
a distally-extending protrusion configured to be seated in a proximal-facing cavity of the surgical implement and having a cross-sectional shape that is one of D-shaped, ovular, Z-shaped, rectangular, star-shaped, hexagonal, or octagonal, and
a distal-facing cavity configured to receive therein a proximally-extending protrusion of the surgical implement and having a cross-sectional shape that is one of D-shaped, ovular, Z-shaped, rectangular, star-shaped, hexagonal, or octagonal.

28. The system of claim 27, wherein the mating feature is configured to cooperate with the mating element to prevent rotation of the surgical implement relative to the adapter with the surgical implement releasably attached to the adapter.

29. The system of claim 27, wherein the mating feature is integrally formed with the adapter.

30. The system of claim 27, wherein the mating feature is configured to releasably attach to the adapter.

31. The system of claim 27, wherein a proximal portion of the adapter is configured to be received within the surgical impacting tool handpiece; and
the mating feature is on a distal-facing surface of the first adapter.

32. The system of claim 27, further comprising the surgical implement.

33. The system of claim 27, wherein the adapter is configured to releasably attach to the surgical impacting tool handpiece.

34. The system of claim 33, further comprising at least one additional adapter;
wherein each of the at least one additional adapters is configured to releasably attach to the surgical impacting tool handpiece such that only one of the adapter and the at least one additional adapter is configured to extend distally from the surgical impacting tool handpiece at a time; and
each of the at least one additional adapters includes a second mating feature configured to releasably couple to a second mating element of a second surgical implement configured to impact bone, the second mating feature is configured to cooperate with the second mating element to prevent rotation of the second surgical implement relative to the additional adapter with the second surgical implement releasably attached to the second adapter.

35. The system of claim 27, wherein the adapter is non-releasably attached to the surgical impacting tool handpiece.

36. The system of claim 27, wherein the mating feature of the adapter is configured to releasably couple to a mating element of a second adapter instead of to the mating element of the surgical implement configured to impact bone;
the mating feature of the adapter is configured to cooperate with the mating element to prevent rotation of the second adapter relative to the adapter with the second adapter releasably attached to the adapter; and
the second adapter is non-releasably coupled to a surgical implement or is configured to releasably couple to a surgical implement.

37. The system of claim 36, further comprising the second adapter.

38. The system of claim 27, further comprising a second adapter configured to releasably attach to the mating feature of the adapter instead of the surgical implement being releasably coupled to the mating feature.

39. The system of claim 38, wherein the second adapter includes a second mating feature configured to releasably couple to a second mating element of a second surgical implement configured to impact bone; and
the second mating feature is configured to cooperate with the second mating element to prevent rotation of the second surgical implement relative to the second adapter with the second surgical implement releasably attached to the second adapter.

40. The system of claim 38, wherein the second adapter is non-releasably coupled to a second surgical implement.

41-43. (canceled)

44. A surgical system, comprising:
a first adapter configured to extend distally from a surgical impacting tool handpiece configured to drive impacting of bone; and
a second adapter configured to extend distally from the first adapter;
wherein the first adapter includes a first mating feature configured to releasably couple to a second mating element of the second adapter; and
the first mating feature is one of:
a distal-facing cavity having a cross pin extending across a diameter of the cavity,
a distally-extending protrusion configured to be seated in a proximal-facing cavity of the surgical implement and having a cross-sectional shape that is one of D-shaped, ovular, Z-shaped, rectangular, star-shaped, hexagonal, or octagonal, and
a distal-facing cavity configured to receive therein a proximally-extending protrusion of the surgical implement and having a cross-sectional shape that is one of D-shaped, ovular, Z-shaped, rectangular, star-shaped, hexagonal, or octagonal.

45. The system of claim 44, wherein the second adapter is non-releasably coupled to a surgical implement configured to impact bone.

46. The system of claim 44, further comprising a surgical implement configured to impact bone;
wherein the second adapter is configured to releasably couple to the surgical implement.

47. The system of claim 44, further comprising a surgical implement configured to impact bone;
wherein the first mating feature of the first adapter is configured to releasably couple to a selected one of the surgical implement and the second adapter.

48. The system of claim 44, wherein the first mating feature is configured to cooperate with the second mating element to prevent rotation of the second adapter relative to the first adapter with the first mating feature releasably coupled to the second mating element.

49-53. (canceled)

54. The system of claim 44, further comprising the surgical impacting tool handpiece.

55-57. (canceled)
